(19) United States
(12) Patent Application Publication
(10) Pub. No.: US 2009/0005442 A1
(43) Pub. Date: Jan. 1, 2009

(54) CARBAMATE COMPOUNDS FOR USE IN PREVENTING OR TREATING NEURODEGENERATIVE DISORDERS

(76) Inventors: Carlos R. Plata-Salaman, Ambler, PA (US); Boyu Zhao, Lansdale, PA (US); Roy E. Twyman, Doylestown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/207,816

(22) Filed: Sep. 10, 2008

Related U.S. Application Data
(62) Division of application No. 10/797,795, filed on Mar. 10, 2004.
Application No. 10/797,795 is a continuation of application No. 10/081,764, filed on Feb. 21, 2002, now abandoned.
(60) Provisional application No. 60/271,682, filed on Feb. 27, 2001.

(51) Int. Cl. A61K 31/27; A61P 25/00

(52) U.S. Cl. 514/483; 514/489

(57) ABSTRACT

This invention is directed to a method for preventing or treating neurodegenerative disorders comprising administering to a subject in need thereof a therapeutically effective amount of a compound selected from the group consisting of Formula (I) and Formula (II):

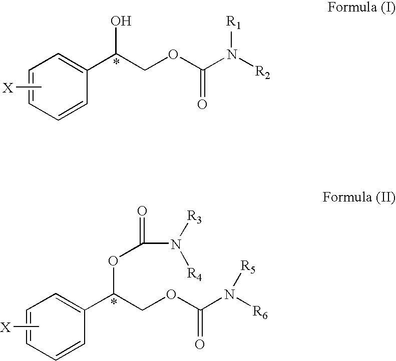

wherein phenyl is substituted at X with one to five halogen atoms selected from the group consisting of fluorine, chlorine, bromine and iodine; and, R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

### CROSS REFERENCE TO RELATED APPLICATIONS

This application is a divisional of application Ser. No. 10/797,795 filed Mar. 10, 2004, which is a continuation of application Ser. No. 10/081,764 filed on Feb. 21, 2002, now abandoned which claims priority of the benefits of the filing of U.S. Provisional Application Ser. No. 60/271,682, filed Feb. 27, 2001. The complete disclosures of the aforementioned related U.S. patent applications are hereby incorporated herein by reference for all purposes.

### FIELD OF THE INVENTION

This invention is directed to a method for use of a carbamate compound in preventing or treating neurodegenerative disorders. More particularly, this invention is directed to a method for use of halogenated 2-phenyl-1,2-ethanediol monocarbamate or dicarbamate compounds for preventing or treating neurodegenerative disorders.

### BACKGROUND OF THE INVENTION

Acute and chronic neurodegenerative disorders are associated with neuronal cell death or compromise (McDonald E S, Windebank A J, Mechanisms of neurotoxic injury and cell death, Neurol. Clin., 2000, August, 18(3), 525-40; Nagy Z, Mechanisms of neuronal death in Down's syndrome, J. Neural. Transm. Suppl, 1999, 57, 233-45; Kilpatrick T J, Soilu-Hanninen M, Molecular mechanisms regulating motor neuron development and degeneration, Mol. Neurobiol., 1999, June, 19(3), 205-28; Rubin L L, Neuronal cell death: an updated view, Prog. Brain. Res., 1998, 117, 3-8; Saha A R, Ninkina N N, Hanger D P, Anderton B H, Davies A M, Buchman V L, Induction of neuronal death by alpha-synuclein, Eur. J. Neurosci., 2000, August, 12(8), 3073-3077; Varadarajan S, Yatin S, Aksenova M, Butterfield D A, Review: Alzheimer's amyloid beta-peptide-associated free radical oxidative stress and neurotoxicity, J. Struct. Biol., 2000, June, 130(2-3), 184-208; Clarke G, Collins R A, Leavitt B R, Andrews D F, Hayden M R, Lumsden C J, McInnes R R, A one-hit model of cell death in inherited neuronal degenerations, Nature, 2000, July, 13, 406(6792), 195-9; Foley P, Riederer P, Influence of neurotoxins and oxidative stress on the onset and progression of Parkinson's disease, J. Neurol., 2000, April, 247 Suppl 2, 1182-94; Nicotera P, Caspase requirement for neuronal apoptosis and neurodegeneration, IUBMB Life, 2000, May, 49(5), 421-5; Mattson M P, Pedersen W A, Duan W, Culmsee C, Camandola S, Cellular and molecular mechanisms underlying perturbed energy metabolism and neuronal degeneration in Alzheimer's and Parkinson's diseases, Ann. N.Y. Acad. Sci., 1999, 893, 154-75; Martin L J, Al-Abdulla N A, Brambrink A M, Kirsch J R, Sieber F E, Portera-Cailliau C, Neurodegeneration in excitotoxicity, global cerebral ischemia, and target deprivation: A perspective on the contributions of apoptosis and necrosis, Brain Res. Bull., 1998, Jul. 1, 46(4), 281-309; Mcintosh T K, Saatman K E, Raghupathi R, Graham D I, Smith D H, Lee V M, Trojanowski J Q, The Dorothy Russell Memorial Lecture; The molecular and cellular sequelae of experimental traumatic brain injury: pathogenetic mechanisms, Neuropathol. Appl. Neurobiol., 1998, August, 24(4), 251-67). Prevention of neuronal cell death is required for the treatment of both acute and chronic neurodegenerative disorders.

Acute neurodegenerative disorders are those associated with an abrupt insult including, but not limited to, acute injury, hypoxia-ischemia or the combination thereof resulting in neuronal cell death or compromise. Acute injury includes, and is not limited to, brain trauma, focal brain trauma, diffuse brain damage, spinal cord injury, intracranial or intravertebral lesions (including, but not limited to, contusion, penetration, shear, compression or laceration lesions) or whiplash shaken infant syndrome. Hypoxia-ischemia includes, and is not limited to, cerebrovascular insufficiency, cerebral ischemia or cerebral infarction (including cerebral ischemias or infarctions originating from embolic occlusion and thrombotic occlusion, reperfusion following acute ischemia, perinatal hypoxic-ischemic injury, cardiac arrest or intracranial hemorrhage of any type (including, but not limited to, epidural, subdural, subarachnoid or intracerebral hemorrhage).

Chronic neurodegenerative disorders are those associated with progressive neuronal cell death or compromise over a period of time including, but not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases (amyotrophic lateral sclerosis), multiple sclerosis, degenerative ataxias, cortical basal degeneration, ALS—Parkinson's—Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease or spinocerebellar ataxia type 3 and olivopontocerebellar degenerations, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome) or prion diseases (including, but not limited to Creutzfeldt-Jakob disease, Gerstmann-Sträussler-Scheinker disease, Kuru disease or fatal familial insomnia).

Other acute or chronic neurodegenerative disorders associated with memory loss include, and are not limited to, neurodegenerative disorders associated with age-related dementia, vascular dementia, diffuse white matter disease (Binswanger's disease), dementia of endocrine or metabolic origin, dementia of head trauma and diffuse brain damage, dementia pugilistica or frontal lobe dementia.

Other acute or chronic neurodegenerative disorders associated with neuronal injury include, and are not limited to, neurodegenerative disorders associated with chemical, toxic, infectious and radiation injury of the nervous system, injury during fetal development, prematurity at time of birth, anoxic-ischemia, injury from hepatic, glycemic, uremic, electrolyte and endocrine origin, injury of psychiatric origin (including, but not limited to, psychopathology, depression or anxiety), injury from peripheral diseases and plexopathies (including plexus palsies) or injury from neuropathy (including neuropathy selected from multifocal, sensory, motor, sensory-motor, autonomic, sensory-autonomic or demyelinating neuropathies (including, but not limited to Guillain-Barre syndrome or chronic inflammatory demyelinating polyradiculoneuropathy) or those neuropathies originating from infections, inflammation, immune disorders, drug abuse, pharmacological treatments, toxins, trauma (including, but not limited to compression, crush, laceration or segmentation traumas), metabolic disorders (including, but not limited to, endocrine or paraneoplastic), Charcot-Marie-Tooth disease (including, but not limited to, type 1a, 1b, 2, 4a or 1-X linked), Friedreich's ataxia, metachromatic leukodystrophy, Refsum's disease, adrenomyeloneuropathy, Ataxia-telangiectasia, Déjerine-Sottas (including, but not limited to, types A or B), Lambert-Eaton syndrome or disorders of the cranial nerves).

Substituted phenyl alkyl carbamate compounds have been described in U.S. Pat. No. 3,265,728 to Bossinger, et al (hereby incorporated by reference), as useful in treating the central nervous system, having tranquilization, sedation and muscle relaxation properties of the formula:

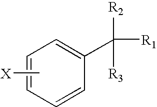

wherein R₁ is either carbamate or alkyl carbamate containing from 1 to 3 carbon atoms in the alkyl group; R₂ is either hydrogen, hydroxy, alkyl or hydroxy alkyl containing from 1 to 2 carbons; R₃ is either hydrogen or alkyl containing from 1 to 2 carbons; and X can be halogen, methyl, methoxy, phenyl, nitro or amino.

A method for inducing calming and muscle relaxation with carbamates has been described in U.S. Pat. No. 3,313,692 to Bossinger, et al (hereby incorporated by reference) by administering a compound of the formula:

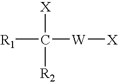

in which W represents an aliphatic radical containing less than 4 carbon atoms, wherein R₁ represents an aromatic radical, R₂ represents hydrogen or an alkyl radical containing less than 4 carbon atoms, and X represents hydrogen or hydroxy or alkoxy and alkyl radicals containing less than 4 carbon atoms or the radical:

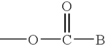

in which B represents an organic amine radical of the group consisting of heterocyclic, ureido and hydrazino radicals and the radical —N(R₃)₂ wherein R₃ represents hydrogen or an alkyl radical containing less than 4 carbon atoms.

Optically pure forms of halogen substituted 2-phenyl-1,2-ethanediol monocarbamates and dicarbamates have also been described in U.S. Pat. No. 6,103,759 to Choi, et al (hereby incorporated by reference), as effective for treating and preventing central nervous system disorders including convulsions, epilepsy, stroke and muscle spasm; and as useful in the treatment of central nervous system diseases, particularly as anticonvulsants, antiepileptics, neuroprotective agents and centrally acting muscle relaxants, of the formulae:

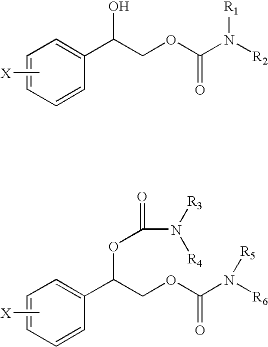

wherein one enantiomer predominates and wherein the phenyl ring is substituted at X with one to five halogen atoms selected from fluorine, chlorine, bromine or iodine atoms and R₁, R₂, R₃, R₄, R₅ and R₆ are each selected from hydrogen and straight or branched alkyl groups with one to four carbons optionally substituted with a phenyl group with substituents selected from the group consisting of hydrogen, halogen, alkyl, alkyloxy, amino, nitro and cyano. Pure enantiomeric forms and enantiomeric mixtures were described wherein one of the enantiomers predominates in the mixture for the compounds represented by the formulae above; preferably one of the enantiomers predominates to the extent of about 90% or greater; and, most preferably, about 98% or greater.

Halogen substituted 2-phenyl-1,2-ethanediol carbamate compounds of Formula (I) or Formula (II) have not been previously described as useful for preventing or treating neurodegenerative disorders. Recent preclinical studies have revealed previously unrecognized pharmacological properties which suggest that a compound of Formula (I) or Formula (II) is useful in preventing or treating neurodegenerative disorders. Therefore, it is an object of the present invention to teach a method for use of a compound of Formula (I) or Formula (II) in preventing or treating neurodegenerative disorders.

### SUMMARY OF THE INVENTION

The present invention is directed to a method for preventing or treating neurodegenerative disorders comprising administering to a subject in need thereof a therapeutically effective amount of a compound selected from the group consisting of Formula (I) and Formula (II):

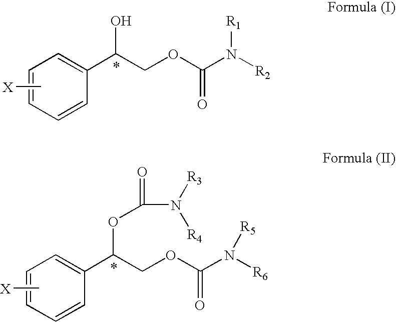

wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

Embodiments of the invention include a method for preventing or treating neurodegenerative disorders comprising administering to a subject in need thereof a therapeutically effective amount of a pharmaceutical composition comprising a pharmaceutically acceptable carrier and a compound selected from the group consisting of Formula (I) and Formula (II).

Embodiments of the invention include the use of a compound selected from the group consisting of Formula (I) and Formula (II) for the preparation of a medicament for preventing or treating neurodegenerative disorders in a subject in need thereof.

Embodiments of the method include the use of an enantiomer selected from the group consisting of Formula (I) and Formula (II) or enantiomeric mixture wherein one enantiomer selected from the group consisting of Formula (I) and Formula (II) predominates. For enantiomeric mixtures wherein one enantiomer selected from the group consisting of Formula (I) and Formula (II) predominates, preferably, one enantiomer selected from the group consisting of Formula (I) and Formula (II) predominates to the extent of about 90% or greater. More preferably, one enantiomer selected from the group consisting of Formula (I) and Formula (II) predominates to the extent of about 98% or greater.

### DETAILED DESCRIPTION OF THE INVENTION

The present invention is directed to a method for preventing or treating neurodegenerative disorders comprising administering to a subject in need thereof a therapeutically effective amount of a compound selected from the group consisting of Formula (I) and Formula (II):

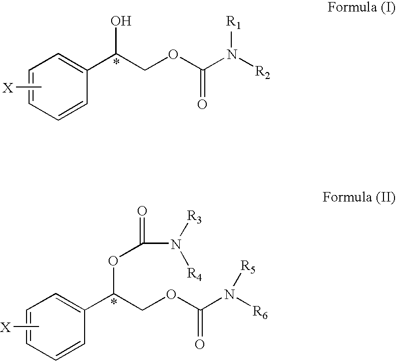

wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

The present method includes the use of a compound selected from the group consisting of Formula (I) and Formula (II) wherein X is chlorine; preferably, X is substituted at the ortho position of the phenyl ring.

The present method also includes the use of a compound selected from the group consisting of Formula (I) and Formula (II) wherein R₁, R₂, R₃, R₄, R₅ and R₆ are preferably selected from hydrogen.

An embodiment of the present method includes the use of an enantiomer selected from the group consisting of Formula (I) and Formula (II) or enantiomeric mixture wherein one enantiomer selected from the group consisting of Formula (I) and Formula (II) predominates wherein X is chlorine; preferably, X is substituted at the ortho position of the phenyl ring.

The present method also includes the use of an enantiomer selected from the group consisting of Formula (I) and Formula (II) or enantiomeric mixture wherein one enantiomer selected from the group consisting of Formula (I) and Formula (II) predominates wherein R₁, R₂, R₃, R₄, R₅ and R₆ are preferably selected from hydrogen.

For enantiomeric mixtures wherein one enantiomer selected from the group consisting of Formula (I) and Formula (II) predominates, preferably, an enantiomer selected from the group consisting of Formula (I) and Formula (II) predominates to the extent of about 90% or greater. More preferably, an enantiomer selected from the group consisting of Formula (I) and Formula (II) predominates to the extent of about 98% or greater.

An embodiment of the present method includes the use of an enantiomer selected from the group consisting of Formula (Ia) and Formula (Ia) or enantiomeric mixture wherein one enantiomer selected from the group consisting of Formula (Ia) and Formula (IIa) predominates:

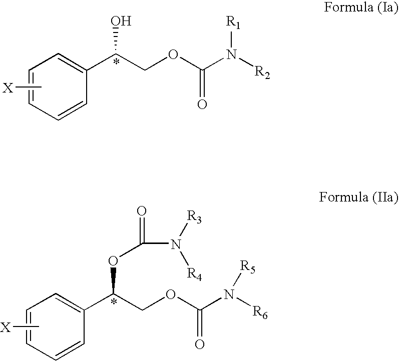

wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

The present method includes the use of an enantiomer selected from the group consisting of Formula (Ia) and Formula (Ia) or enantiomeric mixture wherein one enantiomer selected from the group consisting of Formula (Ia) and Formula (Ia) predominates wherein X is chlorine; preferably, X is substituted at the ortho position of the phenyl ring.

The present method also includes the use of an enantiomer selected from the group consisting of Formula (Ia) and Formula (Ia) or enantiomeric mixture wherein one enantiomer selected from the group consisting of Formula (Ia) and Formula (Ia) predominates wherein R₁, R₂, R₃, R₄, R₅ and R₆ are preferably selected from hydrogen.

For enantiomeric mixtures wherein one enantiomer selected from the group consisting of Formula (Ia) and Formula (Ia) predominates, preferably, an enantiomer selected from the group consisting of Formula (Ia) and Formula (Ia) predominates to the extent of about 90% or greater. More preferably, an enantiomer selected from the group consisting of Formula (Ia) and Formula (Ia) predominates to the extent of about 98% or greater.

An embodiment of the present method includes a method for preventing or treating neurodegenerative disorders comprising administering to a subject in need thereof a therapeutically effective amount of an enantiomer selected from the group consisting of Formula (Ib) and Formula (IIb) or enantiomeric mixture wherein one enantiomer selected from the group consisting of Formula (Ib) and Formula (IIb) predominates:

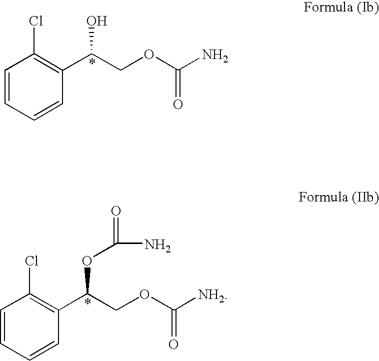

For enantiomeric mixtures wherein one enantiomer selected from the group consisting of Formula (Ib) and Formula (IIb) predominates, preferably, an enantiomer selected from the group consisting of Formula (Ib) and Formula (IIb) predominates to the extent of about 90% or greater. More preferably, an enantiomer selected from the group consisting of Formula (Ib) and Formula (IIb) predominates to the extent of about 98% or greater.

Other crystal forms of the present invention may exist and as such are intended to be included in the present invention.

It is apparent to those skilled in the art that the compounds of the invention are present as racemates, enantiomers and enantiomeric mixtures thereof. A carbamate enantiomer selected from the group consisting of Formula (I), Formula (II), Formula (Ia), Formula (Ia), Formula (Ib) and Formula (IIb) contains an asymmetric chiral carbon atom at the benzylic position, which is the aliphatic carbon adjacent to the phenyl ring (represented by the asterisk in the structural formulae).

Compounds of the present invention may be prepared as described in the previously referenced Bossinger '728 patent (incorporated by reference), Bossinger '692 patent (incorporated by reference) and Choi '759 patent (incorporated by reference).

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

The present invention contemplates a method for preventing or treating neurodegenerative disorders in a subject in need thereof. Neurodegenerative disorders include, and are not limited to, acute neurodegenerative disorders, chronic neurodegenerative disorders, other acute or chronic neurodegenerative disorders associated with memory loss or other acute or chronic neurodegenerative disorders associated with neuronal injury.

Acute neurodegenerative disorders are those associated with an abrupt insult including, but not limited to, acute injury, hypoxia-ischemia or the combination thereof resulting in neuronal cell death or compromise. Acute injury includes, and is not limited to, brain trauma, focal brain trauma, diffuse brain damage, spinal cord injury, intracranial or intravertebral lesions (including, but not limited to, contusion, penetration, shear, compression or laceration lesions) or whiplash shaken infant syndrome. Hypoxia-ischemia includes, and is not limited to, cerebrovascular insufficiency, cerebral ischemia or cerebral infarction (including cerebral ischemias or infarctions originating from embolic occlusion or thrombotic occlusion, reperfusion following acute ischemia, perinatal hypoxic-ischemic injury, cardiac arrest or intracranial hemorrhage of any type (including, but not limited to, epidural, subdural, subarachnoid or intracerebral hemorrhage)).

Chronic neurodegenerative disorders are those associated with progressive neuronal cell death or compromise over a period of time including, but not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases (amyotrophic lateral sclerosis), multiple sclerosis, degenerative ataxias, cortical basal degeneration, ALS—Parkinson's—Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome) or prion diseases (including, but not limited to Creutzfeldt-Jakob disease, Gerstmann-Sträussler-Scheinker disease, Kuru disease or fatal familial insomnia).

Other acute or chronic neurodegenerative disorders associated with memory loss include, and are not limited to, neurodegenerative disorders associated with age-related dementia, vascular dementia, diffuse white matter disease (Binswanger's disease), dementia of endocrine or metabolic origin, dementia of head trauma and diffuse brain damage, dementia pugilistica or frontal lobe dementia.

Other acute or chronic neurodegenerative disorders associated with neuronal injury include, and are not limited to, neurodegenerative disorders associated with chemical, toxic, infectious and radiation injury of the nervous system, injury during fetal development, prematurity at time of birth, anoxic-ischemia, injury from hepatic, glycemic, uremic, electrolyte and endocrine origin, injury of psychiatric origin (including, but not limited to, psychopathology, depression or anxiety), injury from peripheral diseases and plexopathy (including plexus palsies) or injury from neuropathy (including neuropathy selected from multifocal, sensory, motor, sensory-motor, autonomic, sensory-autonomic or demyelinating neuropathies (including, but not limited to, Guillain-Barre syndrome or chronic inflammatory demyelinating polyradiculoneuropathy) or those neuropathies originating from infections, inflammation, immune disorders, drug abuse, pharmacological treatments, toxins, trauma (including, but not limited to, compression, crush, laceration or segmentation traumas), metabolic disorders (including, but not limited to, endocrine or paraneoplastic), Charcot-Marie-Tooth disease (including, but not limited to, type 1a, 1b, 2, 4a or 1-X linked), Friedreich's ataxia, metachromatic leukodystrophy, Refsum's disease, adrenomyeloneuropathy, Ataxia-telangiectasia, Déjerine-Sottas (including, but not limited to, types A or B), Lambert-Eaton syndrome or disorders of the cranial nerves).

An example of the method of the present invention comprises administering to the subject a therapeutically effective amount of a compound selected from the group consisting of Formula (I) and Formula (II) in a pharmaceutical composition comprising a pharmaceutically acceptable carrier and a compound selected from the group consisting of Formula (I) and Formula (II). The method of the present invention also includes the use of a compound selected from the group consisting of Formula (I) and Formula (II) for the preparation of a medicament for preventing or treating neurodegenerative disorders.

Another example of the method of the present invention comprises administering to the subject a therapeutically effective amount of a compound selected from the group consisting of Formula (I) and Formula (II) or a pharmaceutical composition thereof in combination with one or more agents useful in preventing or treating neurodegenerative disorders.

A compound selected from the group consisting of Formula (I) and Formula (II) or pharmaceutical composition thereof may be administered by any conventional route of administration including, but not limited to oral, pulmonary, intraperitoneal (ip), intravenous (iv), intramuscular (im), subcutaneous (sc), transdermal, buccal, nasal, sublingual, ocular, rectal and vaginal. In addition, administration directly to the nervous system may include, and are not limited to, intracerebral, intraventricular, intracerebroventricular, intrathecal, intracisternal, intraspinal or peri-spinal routes of administration by delivery via intracranial or intravertebral needles or catheters with or without pump devices. It will be readily apparent to those skilled in the art that any dose or frequency of administration that provides the therapeutic effect described herein is suitable for use in the present invention.

The therapeutically effective amount of a compound selected from the group consisting of Formula (I) and Formula (II) or pharmaceutical composition thereof may be from about 0.01 mg/Kg/dose to about 100 mg/Kg/dose. Preferably, the therapeutically effective amount may be from about 0.01 mg/Kg/dose to about 25 mg/Kg/dose. More preferably, the therapeutically effective amount may be from about 0.01 mg/Kg/dose to about 10 mg/Kg/dose. Most preferably, the therapeutically effective amount may be from about 0.01 mg/Kg/dose to about 5 mg/Kg/dose. Therefore, the therapeutically effective amount of the active ingredient contained per dosage unit (e.g., tablet, capsule, powder, injection, suppository, teaspoonful and the like) as described herein may be from about 1 mg/day to about 7000 mg/day for a subject, for example, having an average weight of 70 Kg.

The dosages, however, may be varied depending upon the requirement of the subjects (including factors associated with the particular subject being treated, including subject age, weight and diet, strength of the preparation, the advancement of the disease condition and the mode and time of administration) and the use of a particular compound of Formula (I) or Formula (II) or pharmaceutical composition thereof.

Optimal dosages to be administered may be readily determined by those skilled in the art and will result in the need to adjust the dose to an appropriate therapeutic level. The use of either daily administration or post-periodic dosing may be employed. Preferably, a compound of Formula (I) or Formula (II) or pharmaceutical composition thereof for preventing or treating neurodegenerative disorders is administered orally or parenterally.

In accordance with the methods of the present invention, a compound of Formula (I) or Formula (II) or pharmaceutical composition thereof described herein may be administered separately, at different times during the course of therapy or concurrently in divided combination or single combination forms. Advantageously, a compound selected from the group consisting of Formula (I) and Formula (II) or pharmaceutical compositions thereof may be administered in a single daily dose or the total daily dosage may be administered via continuous delivery or in divided doses of two, three or four times daily. The instant invention is therefore to be understood as embracing all such methods and regimes of continuous, simultaneous or alternating treatment and the term “administering” is to be interpreted accordingly.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human, that is being sought by a researcher, veterinarian, medical doctor, or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

To prepare a pharmaceutical composition of the present invention, a compound of Formula (I) or Formula (II) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Preferably, a pharmaceutical composition is in a unit dosage form such as a tablet, pill, capsule, caplet, gelcap, lozenge, granule, powder, sterile parenteral solution or suspension, metered aerosol or liquid spray, drop, ampoule, autoinjector device or suppository for administration by oral, intranasal, sublingual, intraocular, transdermal, parenteral, rectal, vaginal, inhalation or insufflation means. Alternatively, the composition may be presented in a form suitable for once-weekly or once-monthly administration or may be adapted to provide a preparation for intramuscular injection.

In preparing a pharmaceutical composition having a solid dosage form for oral administration, such as a tablet, pill, capsule, caplet, gelcap, lozenge, granule or powder (each including immediate release, timed release and sustained release formulations), suitable carriers and additives include but are not limited to diluents, granulating agents, lubricants, binders, glidants, disintegrating agents and the like. If desired, tablets may be sugar coated, gelatin coated, film coated or enteric coated by standard techniques.

For preparing a solid dosage form, the principal active ingredient is mixed with a pharmaceutical carrier (e.g. conventional tableting ingredients such as diluents, binders, adhesives, disintegrants, lubricants, antiadherents and glidants). Sweeteners and flavorants may be added to chewable solid dosage forms to improve the palatability of the oral dosage form. Additionally, colorants and coatings may be added or applied to the solid dosage form for ease of identification of the drug or for aesthetic purposes. These carriers are formulated with the pharmaceutical active to provide an accurate, appropriate dose of the pharmaceutical active with a therapeutic release profile.

In preparing a pharmaceutical composition having a liquid dosage form for oral, topical and parenteral administration, any of the usual pharmaceutical media or excipients may be employed. Thus, for liquid unit dosage forms, such as suspensions (i.e. colloids, emulsions and dispersions) and solutions, suitable carriers and additives include but are not limited to pharmaceutically acceptable wetting agents, dispersants, flocculation agents, thickeners, pH control agents (i.e. buffers), osmotic agents, coloring agents, flavors, fragrances, preservatives (i.e. to control microbial growth, etc.) and a liquid vehicle may be employed. Not all of the components listed above will be required for each liquid dosage form. The liquid forms in which the novel compositions of the present invention may be incorporated for administration orally or by injection include, but are not limited to aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles.

### BIOLOGICAL EXPERIMENTAL EXAMPLES

The activities of a compound of Formula (I) and Formula (II) for use in preventing or treating neurodegenerative disorders were evaluated in the following experimental example which is intended to be a way of illustrating but not limiting the invention.

### Example 1

### PC12 Cell Serum Withdrawal Model

Serum withdrawal is a cytotoxic environmental challenge that results in cell death in cultured cell lines as well as in primary cells of various tissue origins, including nerve cells. In particular, pheochromocytoma (PC) 12 cells have been widely employed as an in vitro neuronal cell model for a wide variety of neurodegenerative and cell death related disorders (Muriel, et al, Mitochondrial free calcium levels (Rhod-2 fluorescence) and ultrastructural alterations in neuronally differentiated PC12 cells during ceramide-dependent cell death, J. Comp. Neurol., 2000, 426(2), 297-315; Dermitzaki, et al, Opioids transiently prevent activation of apoptotic mechanisms following short periods of serum withdrawal, J. Neurochem., 2000, 74(3), 960-969; Carlile, et al, Reduced apoptosis after nerve growth factor and serum withdrawal: conversion of tetrameric glyceraldehyde-3-phosphate dehydrogenase to a dimer, Mol. Pharmacol., 2000, 57(1), 2-12).

PC12 cells were cultured in sterile media (RPMI 1640) supplemented with 10% heat-inactivated horse serum and 5% fetal bovine serum (FBS). The culture medium also contained 1× Penicillin-Streptomycin-Neomycin antibiotic (50 μg, 50 μg, 100 μg, respectively). Medium was exchanged every other day and the cells were passed in log phase near confluence.

The control cells were cultured in regular media without any treatment. An enantiomer of Formula (Ib) or Formula (Ib) (10 μM) was mixed well in the medium and then applied to the cells. For the 2 day assay, an enantiomer of Formula (Ib) or Formula (Ib) (10 μM) was only applied to the cells once at the time of serum withdrawal. For the 7 day assay, an enantiomer of Formula (Ib) or Formula (IIb) (10 μM) was applied to the cells at the time of serum withdrawal and every 48 hr thereafter when cells were changed with fresh new serum-free medium. In the serum withdrawal group, the cells were cultured in serum-free medium with no additional enantiomer of Formula (Ib) or Formula (IIb). Cell survival was determined by the 3-(4,5-dimethylthiazol-2-yl)-5-(3-carboxy-methoxyphenyl)-2-(4-sulfophenyl)-2H-tetrazolium inner salt (MTS) assay at 2 or 7 days after serum withdrawal.

At the end of the experiment, cells were washed with fresh medium and incubated with MTS solution in a humidified 37° C. with 5% CO2 incubator for 1.5 hr. After the incubation period, the cells were immediately analyzed using a Softmax program (Molecular Devices). MTS assay is a calorimetric method for determining the number of viable cells in a given experimental setting. The assay is based on the cellular conversion of the tetrazolium salt, MTS, into a formazan that is soluble in tissue culture medium and measured directly at 490 nm in 96-well assay plates. The absorbance is directly proportional to the number of living cells in culture. The arbitrary absorbance reading in control cells is expressed as 100% survival rate.

Table 1 lists data demonstrating the effect on cell survival rate of the orally administered enantiomer of Formula (Ib) and Formula (IIb) in the PC 2 cell serum withdrawal model (¹p value=0.01; ²p value=<0.01).

TABLE 1
% Cell Survival Rate

| | 2 Day | 7 Day |
|---|---|---|
| | Survival Rate (%) | Survival Rate (%) |
| Control | 100 | 100 |
| Serum-free | 49.6 ± 2.6 | 23.8 ± 2.6 |
| Formula (Ib) | 69.4 ± 1.7¹ | 79.9 ± 4.0² |
| Formula (IIb) | 66.4 ± 5.4¹ | 85.2 ± 0.6² |

### Example 2

### The Transient Cerebral Ischemia Rat Model

An enantiomer of Formula (Ib) was investigated in the transient cerebral ischemia middle cerebral artery occlusion (MCAO) rat model (as described in Nagasawa H. and Kogure K ., Stroke, 1989, 20, 1037; and, Zea Longa E., Weinstein P. R., Carlson S, and Cummins R., Stroke, 1989, 20, 84) using male Wistar rats at 10 and 100 mg/kg (i.v.). MK 801 (Dizocilpine maleate; CAS Registry number 77086-22-7, a commercially available compound) was used as a positive control (3 mg/kg, i.p.).

Rats (n=12) were randomly allocated to one of four experimental groups and were anesthetized. Blood flow from the internal carotid artery, anterior cerebral artery and posterior cerebral artery into the middle cerebral artery was blocked by this procedure. One hour after blockage, animals were treated over a 1 hour period with vehicle (administered i.v. over the one hour period), control (administered as a single i.p. dose at the start of the one hour period) and two doses of an enantiomer of Formula (Ib) (administered i.v. over the one hour period). Two hours after blockage, reperfusion was performed.

The animals were sacrificed and 20 mm-thick coronal sections of each brain were prepared. One in every forty sections (i.e. every 800 nM) from the front to the occipital cortex was used to quantify the extent of the cerebral lesion. Slides were prepared using sections stained (according to the Nissl procedure) with cresyl violet and were examined under a light microscope.

Regional ischemic surface areas in the coronal sections of individual rats were determined according to the presence of cells with morphological changes. The areas of neuronal injury or infarction were measured and then added. The cortex and striatum volume were calculated for each animal (total ischemic surface area×0.8 mm (thickness)).

### MCAO Model Analysis

The mean volumes (±S.E.M.) for each animal randomly assigned to the four experimental groups were compared using one-way ANOVA (one way ANOVA is a statistical method which compares 3 or more unmatched groups) followed by Dunnett's t-test (both methods incorporated in Statview 512+ software, BarinPower, Calabasas, Calif., USA).

As shown in Table 2, results were considered statistically significant when the p value was <0.05 compared to vehicle group (₁p<0.01; ²p<0.05).

TABLE 2

| | | Mean Infarct Volume (mm³) ± S.E.M. | | |
|---|---|---|---|---|
| Treatment | N | Cortex | Striatum | Total Volume |
| Vehicle, 10 mL/kg | 12 | 275.5 ± 27.1 | 79.4 ± 3.6 | 354.9 ± 29.9 |
| MK 801, 3 mg/kg | 12 | 95.8 ± 24.5¹ | 56.1 ± 5.3² | 151.9 ± 28.7¹ |
| Formula (Ib), | 12 | 201.0 ± 23.9 | 75.9 ± 2.6 | 276.9 ± 25.4 |
| 10 mg/kg | | | | |
| Formula (Ib), | 12 | 98.8 ± 29.5¹ | 63.0 ± 5.9² | 161.9 ± 34.3¹ |
| 100 mg/kg | | | | |

While the foregoing specification teaches the principles of the present invention, with examples provided for the purposes of illustration, it will be understood that the practice of the invention encompasses all of the usual variations, adaptations and/or modifications as come within the scope of the following claims and their equivalents.

1. A method for preventing or treating neurodegenerative disorders comprising administering to a subject in need thereof a therapeutically effective amount of a compound selected from the group consisting of Formula (I) and Formula (II): 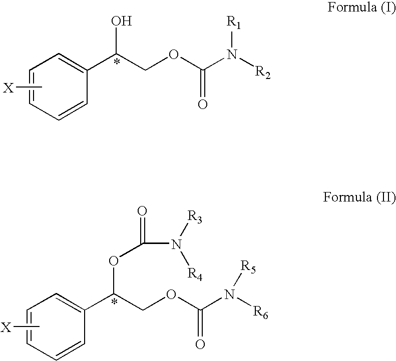
wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

2. The method of claim 1 wherein X is chlorine.

3. The method of claim 1 wherein X is substituted at the ortho position of the phenyl ring.

4. The method of claim 1 wherein R₁, R₂, R₃, R₄, R₅ and R₆ are selected from hydrogen.

5. A method for preventing or treating neurodegenerative disorders comprising administering to a subject in need thereof a therapeutically effective amount of an enantiomer selected from the group consisting of Formula (I) and Formula (II) or enantiomeric mixture wherein one enantiomer selected from the group consisting of Formula (I) and Formula (II) predominates: 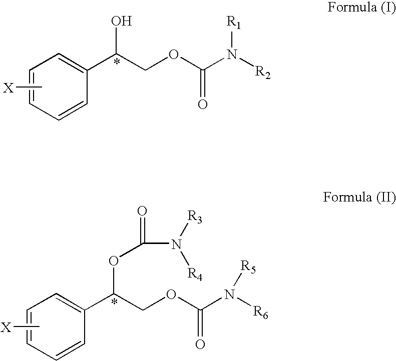
wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

6. The method of claim 5 wherein X is chlorine.

7. The method of claim 5 wherein X is substituted at the ortho position of the phenyl ring.

8. The method of claim 5 wherein R₁, R₂, R₃, R₄, R₅ and R₆ are selected from hydrogen.

9. The method of claim 5 wherein one enantiomer selected from the group consisting of Formula (I) and Formula (II) predominates to the extent of about 90% or greater.

10. The method of claim 5 wherein one enantiomer selected from the group consisting of Formula (I) and Formula (II) predominates to the extent of about 98% or greater.

11. The method of claim 5 wherein the enantiomer selected from the group consisting of Formula (I) and Formula (II) is an enantiomer selected from the group consisting of Formula (Ia) and Formula (IIa): 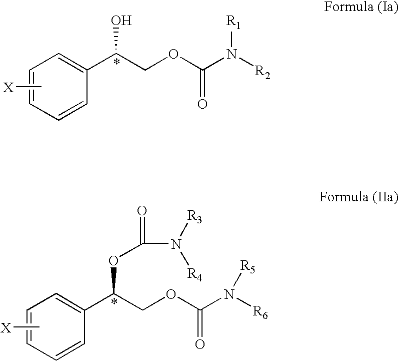
wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

12. The method of claim 11 wherein X is chlorine.

13. The method of claim 11 wherein X is substituted at the ortho position of the phenyl ring.

14. The method of claim 11 wherein R₁, R₂, R₃, R₄, R₅ and R₆ are selected from hydrogen.

15. The method of claim 11 wherein one enantiomer selected from the group consisting of Formula (Ia) and Formula (IIa) predominates to the extent of about 90% or greater.

16. The method of claim 11 wherein one enantiomer selected from the group consisting of Formula (Ia) and Formula (IIa) predominates to the extent of about 98% or greater.

17. The method of claim 5 wherein the enantiomer selected from the group consisting of Formula (I) and Formula (II) is an enantiomer selected from the group consisting of Formula (Ib) and Formula (IIb):

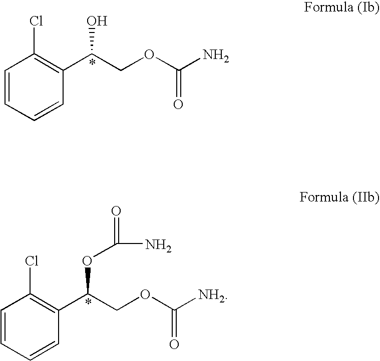

18. The method of claim 17 wherein one enantiomer selected from the group consisting of Formula (Ib) and Formula (IIb) predominates to the extent of about 90% or greater.

19. The method of claim 17 wherein one enantiomer selected from the group consisting of Formula (Ib) and Formula (IIb) predominates to the extent of about 98% or greater.

20. The method as in claims 1 or 5 wherein neurodegenerative disorders are selected from the group consisting of acute neurodegenerative disorders, chronic neurodegenerative disorders, other acute or chronic neurodegenerative disorders associated with memory loss and other acute or chronic neurodegenerative disorders associated with neuronal injury.

21. (canceled)

22. The method of claim 20, wherein acute injury is selected from brain trauma, focal brain trauma, diffuse brain damage, spinal cord injury, intracranial lesions (selected from contusion, penetration, shear, compression or laceration lesions), intravertebral lesions (selected from contusion, penetration, shear, compression or laceration lesions) or whiplash shaken infant syndrome.

23. The method of claim 22 wherein acute injury is selected from brain trauma, focal brain trauma, diffuse brain damage or spinal cord injury.

24. (canceled)

25. (canceled)

26. The method of claim 20 wherein chronic neurodegenerative disorders are selected from neurodegenerative disorders associated with progressive neuronal cell death or compromise over a period of time selected from Alzheimer's disease, Pick's disease, diffuse Lewy body disease, progressive supranuclear palsy (selected from Steel-Richardson syndrome), multisystem degeneration (selected from Shy-Drager syndrome), chronic epileptic conditions associated with neurodegeneration, motor neuron diseases (selected from amyotrophic lateral sclerosis), multiple sclerosis, degenerative ataxias, cortical basal degeneration, ALS—Parkinson's—Dementia complex of Guam, subacute sclerosing panencephalitis, Huntington's disease, Parkinson's disease, synucleinopathies (selected from multiple system atrophy), primary progressive aphasia, striatonigral degeneration, Machado-Joseph disease/spinocerebellar ataxia type 3 and olivopontocerebellar degenerations, bulbar and pseudobulbar palsy, spinal and spinobulbar muscular atrophy (selected from Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (selected from Riley-Day syndrome) or prion diseases (selected from Creutzfeldt-Jakob disease, Gerstmann-Sträussler-Scheinker disease, Kuru disease or fatal familial insomnia).

27. The method of claim 26 wherein chronic neurodegenerative disorders are selected from Alzheimer's disease, chronic epileptic conditions associated with neurodegeneration, multiple sclerosis or Parkinson's disease.

28. The method of claim 20 wherein other acute or chronic neurodegenerative disorders associated with memory loss are selected from neurodegenerative disorders associated with age-related dementia, vascular dementia, diffuse white matter disease (selected from Binswanger's disease), dementia of endocrine or metabolic origin, dementia of head trauma or diffuse brain damage, dementia pugilistica or frontal lobe dementia.

29. The method of claim 20 wherein other acute or chronic neurodegenerative disorders associated with neuronal injury are selected from neurodegenerative disorders associated with chemical, toxic, infectious and radiation injury of the nervous system, injury during fetal development, prematurity at time of birth, anoxic-ischemia, injury from hepatic, glycemic, uremic, electrolyte and endocrine origin, injury of psychiatric origin, injury from peripheral diseases and plexopathy (selected from plexus palsies) or injury from neuropathy.

30. The method of claim 29 wherein other acute or chronic neurodegenerative disorders associated with neuronal injury are selected from neurodegenerative disorders associated with injury of psychiatric origin or injury from neuropathy.

31. The method of claim 30 wherein injury of psychiatric origin is selected from psychopathology, depression or anxiety; and, wherein injury from neuropathy is selected from multifocal, sensory, motor, sensory-motor, autonomic, sensory-autonomic or demyelinating neuropathies (selected from Guillain-Barre syndrome or chronic inflammatory demyelinating polyradiculoneuropathy) or those neuropathies originating from infections, inflammation, immune disorders, drug abuse, pharmacological treatments, toxins, trauma (selected from compression, crush, laceration or segmentation traumas), metabolic disorders (selected from endocrine or paraneoplastic), Charcot-Marie-Tooth disease (selected from type 1a, 1b, 2, 4a or 1-X linked), Friedreich's ataxia, metachromatic leukodystrophy, Refsum's disease, adrenomyeloneuropathy, Ataxia-telangiectasia, Déjerine-Sottas (selected from types A or B), Lambert-Eaton syndrome or disorders of the cranial nerves).

32. The method as in claims 1 or 5 wherein the therapeutically effective amount is from about 0.01 mg/Kg/dose to about 100 mg/Kg/dose.
